(19) United States
(12) Patent Application Publication
(10) Pub. No.: US 2004/0203085 A1
(43) Pub. Date: Oct. 14, 2004

(54) Method for parallel synthesis and transfer of molecules to a substrates

(76) Inventors: Andre Bernard, Tubingen (DE); Stefan Dubel, Dossenheim (DE)

Correspondence Address: NATH & ASSOCIATES, 1030 15th STREET, 6TH FLOOR, WASHINGTON, DC, 20005, US

(21) Appl. No.: 10/475,801

(22) PCT Filed: Apr. 26, 2002
(86) PCT No.: PCT/EP02/04670

(30) Foreign Application Priority Data
Apr. 26, 2001 (DE) 10121574.6

(51) Int. Cl. G01N 33/53

(52) U.S. Cl. 435/7.94

(57) ABSTRACT

The invention relates to a method for highly parallel production of complex molecule libraries on the surface of a substrate with the aid of regeneratable matrix dies. According to prior art, the production of molecule libraries on a support is expensive, and requires a lot of time and a large amount of apparatus. By using regeneratable matrix dies it is possible to simplify and accelerate the production of molecule libraries, especially DNA and protein libraries, in a significant manner. The matrix-10 die is made of an elastic material such as polydimethylsiloxane and includes locally-bound matrix molecules on the surface thereof, said molecules being used as a model for the synthesis of copy molecules. The copy molcules are transferred to the target surface by means of a contact printing method. The matrix molecules remain on the die for further synthesis and print cycles which can repeated several times. The inventive method is suitable for copying and reproducing differently produced molecule arrays.

The present invention relates to a method for highly parallel preparation of molecule libraries on a substrate surface.

The microarray technology in which various biomolecules such as DNA or proteins are applied to a substrate surface in a tightly packed manner and a predefined pattern have now become the standard method for parallel analysis of biological samples. Said technology is used, for example, in the analysis of gene expression, in genetic diagnostics, in biological and pharmaceutical research and for determining genetically engineered organisms in the food industry.

The limiting factor in terms of costs and time for mass production of molecule arrays is the many times reproducible application of molecules to metal, glass, membrane or plastic surfaces. In principle, the following two techniques are known for this method: 1) an in situ synthesis of array molecules from monomers by means of photochemically or electrostatically mediated reactions in situ directly on a support (U.S. Pat. No. 5,405,783); 2) applying ready-made macromolecules either in drop form (spotting) by means of printing pin (U.S. Pat. No. 6,101,946), micropipettes (U.S. Pat. No. 5,601,890) or ink jet printers (U.S. Pat. No. 5,927,547). For the current state of the art, in particular for the use of microarrays in DNA and protein analysis, the reader is referred to the following specialist review articles: Nature Genetics, vol. 21, No. 1 supplement (1999).

When applying macromolecules to the support surface, the diameter of the sites where the molecules are attached to the support (spots) is determined by the nature of the substrate surface and the drop size or the size of the printing pin used for applying said molecules. Typically, spot sizes in the known methods are from 50 μm to 200 μm. Only by applying the photolithographic technique which uses light masks specifically prepared therefore for direct solid-phase synthesis of DNA in situ (WO 92/10092), it is possible to obtain a substantially higher density and smaller spot sizes. However, this method has considerable technical complexity and is time-consuming and expensive.

The known methods for preparing molecule arrays have the disadvantage that preparation of each individual array requires many steps so that the possibility of paralleling the process is only remotely, if at all, possible. The different types of molecules must be applied in each case individually to the array surface, resulting, in the case of many thousands of spots per array, in an immense number of individual steps. This requires a large amount of time, apparatus and costs and generates problems regarding reproducibility.

It is the object of the present invention to develop a method which allows a more rapid and cost-effective preparation of molecule libraries on a substrate surface with higher precision and reproducibility. Moreover, this method should make it possible to achieve a spot size of less than 10 μm, and in particular less than 2 μm, and also a higher spot density than by means of standard methods such as, for example, application in drop form.

To achieve this object, the invention proposes primarily a method having the features as defined in claim 1. Developments of the invention are subject matters of the remaining dependent and independent claims 2 to 33 whose wording as well as the wording of the abstract are incorporated by reference into this specification. The use of the method of the invention renders the preparation of molecule arrays highly parallel and simplifies and accelerates the production process.

The invention describes a method for space-resolved and selective synthesis of complex chemical compounds on a substrate surface, which all together form a dot matrix, a circular, helical, strip-shaped, linear or other geometric of stochastic arrangement of molecules on a substrate surface.

The abovementioned object is achieved according to the invention by using firstly a stamping technique by which the entire substrate surface area or subareas are coated with various biomolecules in a single print process. Secondly, said biomolecules are, in each case in between the stamping processes, synthesized de novo and true to location on the substrate on the basis of a template applied to the die. De-novo synthesis of array molecules on the surface of said die regenerates the latter for the new print process. The die thus not only serves as transport vehicle for transferring the array molecules to the place of destination on the substrate surface but is also a blueprint for a reproducible synthesis of said molecules.

The material used for the die is advantageously plastic, glass or metal. A preferred embodiment of the invention uses a polymer with elastic 5 properties (elastomer) or a combination of various materials, in particular a die with a surface layer made of an elastomer and a core made of a different material. In a particularly preferred embodiment, polydimethylsiloxane (PDMS) is used for the preparation of dies. PDMS is a synthetic-material which can be cast into any shape and cured (Delamarche et al., Advanced Materials 9: 741-746 (1997)). It is particularly suitable as a material for dies, since, owing to its physical properties, it is capable of adapting to the surface to be stamped on with very high precision and thus ensuring uniform contact of the two surfaces.

According to the invention, a die is used whose surface carries an array of template molecules. The template molecules may be connected with or applied to the die surface covalently or noncovalently. The template molecules may be applied to the die mechanically according to the prior art, in particular by application in drop form 20, for example with the aid of ink jet printers or printing pins, or by means of lithographic methods according to the prior art. Alternatively, the die may also be rolled with the template molecules in one or more stamping processes in which, in each case, individual molecule components are assembled to complete macromolecules in a step-by-step synthesis. It is also possible to apply various complete molecules at predefined positions on the die surface by using a congruent set of multiple dies or masks.

Another advantageous variant uses for the preparation of dies a material which is transparent for a particular light wavelength, such as polydimethylsiloxane, for example. According to the invention, the light-conducting properties of the die may be used to couple to or synthesize at the predefined locations particular molecules via location-selective light conduction.

A synthesis controlled by electric fields directly on the die is also possible.

The covalent linkage of the template molecules may be achieved, for example, via binding of already present or specifically introduced amino, thio or phospho groups to a silanized surface with functionalized terminal groups. Alternatively, biotinylated template molecules may be specifically immobilized on the die surface by means of streptavidin coating.

Copy molecules (image molecules) are prepared enzymatically on the template on the die surface in a heterogeneous synthesis according to a standard technique. After synthesis, the copy molecules remain at the location of the particular template molecules on the die. According to the invention, the copy molecules may comprise an anchor by which they may be immobilized at a destination on the target surface.

In one embodiment, the anchor used may be an oligopeptide (“tag”), for example. In this case, the surface to be stamped is provided with a layer of molecules which bind with high affinity to the tag group of the newly synthesized copy molecules, in preparation of the stamping process. This coating may likewise be applied by stamping and may be limited to particular areas of the surface in order to generate position information for the later analytical and selection processes. In particular, the tag may comprise from fifteen to twenty N-terminal amino acids of human pancreatic Rnase which binds with high affinity to a proteolytic fragment of the homologous bovine RNase or to a corresponding fragment of other species. In another typical embodiment, said tag is the epitope peptide of an antibody or of another molecule, for example of streptavidin. Alternatively, for example, biotin may be coupled to the copy molecules, which causes binding with high affinity to the substrate coated with streptavidin or avidin. Other haptenes and their binding proteins, such as PHOX (4-ethoxymethylene-2-phenyl-2-oxazolin-5-one), digoxigenin, FITC and others, may also be used.

The template die loaded with copy molecules is contacted with the target surface. The freshly synthesized copy molecules of the individual spots are transferred by means of a contact printing method. The template molecules remain on the die and are available again for another synthesis of copy molecules on the die surface. After the die has been regenerated, another substrate can be stamped.

Compared to the known methods, the method of the invention has the advantage that a plurality of different types of molecules are transferred to the substrate surface in a single process, enabling each new array to be prepared in a single step. According to the invention, this sequence of processes—regeneration of die by de-novo synthesis, stamping—may be repeated several times. The actual preparation and designing of the template array and the application of the template molecules to the die surface are carried out only once in the method described herein. The finished die then serves as template for the synthesis of a plurality of copy arrays.

In another embodiment of the invention, the method described may be extended in such a way that arrays (referred to as original arrays hereinbelow) prepared via a different route, for example by a standard method, may be copied and duplicated. For this purpose, the original array with array molecules placed thereupon is utilized as template for the synthesis of complementary molecules which serve as templates for the synthesis of copy molecules in further steps. Said template molecules are taken up and bound by a still empty die. Anchor elements which may be attached to the newly synthesized template molecules may promote binding of the latter to the die surface. The template molecules on the die may be used as described above for the steps of reproducible copy synthesis and stamping.

Starting from a copy array prepared according to the method described, one or more further template dies may be generated which in turn are employed in the synthesis of copy arrays. Via iterative synthesis of this kind of template and copy arrays, the template dies may be duplicated to a considerable extent and be used in a production process for industrial 30 production of a large number of molecule arrays.

The invention has a multiplicity of other advantages. Unlike other known methods, the method is not limited to the synthesis of short oligomers. The invention makes it possible to apply highly complex substances and even molecule complexes, including modifications, to a substrate. Moreover, it is possible to prepare arrays with a plurality of, in particular more than 1 000, spots in a definable spatial distribution in a single step. The exact position of each individual molecule spot can be readily determined here via the arrangement of the latter relative to a label on the substrate.

Furthermore, the methods described may be used advantageously in order to prepare arrays with smaller spot sizes and higher spot densities than by known methods, for example, by application in drop form. This may be achieved by the template die having, apart from chemical information in the form of template molecules applied, for example by application in drop form, with a defined spot density to the die surface, additionally topological information in the form of relief structures. The topological structures may be, for example, elevations whose dimensions may be smaller than 1 μm. The basic requirement for using the die in the method of the invention is a conformal contact to the substrate surface. In addition, the structural elements must not sag and not be distorted too much during contact with the substrate surface. The structures are characterized here by the aspect ratio (ratio of the height of the structure to its lateral dimensions in periodic structures) and the filling factor (ratio of the contact area of the structures to the total area) (see also: Delamarche et al., Advanced Materials 9: 741-746 (1997): Bietsch and Michel, J. Appl. Phys. 88: 4310-4318 (2000)). An aspect ratio of from 1:5 to 5:1, in some cases from 1:20 to 20:1 when using suitable materials, is particularly recommendable. The filling factor should be at least 5%-10%.

The cross-sectional area of the elevations is preferably made with smaller dimensions than the area of the spots generated according to the prior art by application in drop form. In a preferred embodiment, the distance between neighboring elevations equals the distance between individual neighboring spots on the die so that the spots are positioned in the same grid as the respective elevations. The spot edges which are normally blurred due to the application method extend beyond the edges of the elevations. After a synthesis, however, only those copy molecules which are located on the protruding proximal smooth area are transferred in the stamping process, since they are the only ones coming into direct contact with the substrate surface.

This leads to a reduction in spot size, compared to the spot size generated by a standard method, for example by application in drop form. In order to obtain a higher spot density than on the die, a plurality of stamping processes is carried out. Prior to each new stamping process, the die is moved by translation or/and rotation relative the die surface so that the new spots are placed between the previously applied spots. In this way, spots can be arranged on the substrate surface in any three-dimensional form.

If, for example, spots need to be applied according to a regular matrix pattern common for microarrays, the substrate surface is moved in each subsequent stamping process along its x- or y-axis relative to the die surface by a defined length b which is shorter than the distance a between the elevations on the die. In this connection, preference is given to using in successive stamping processes dies having the same topology but different coatings. If the distance between the neighboring spots on the die is a and the density is D₁ and if an array with a distance b between the neighboring spots at a density D₂ is to be prepared, then a₂/b₂ (D₂/D₁) different dies and stamping processes are required.

In another variant, a smaller spot size can be obtained by introducing a relief structure on the substrate. Alternatively, relief structures on both surfaces, the die and the substrate, are adjusted to one another so as to generate the desired spot density.

In a another embodiment, the template array for the topological die may be synthesized on molecules of the original array, using dies on which the distance between neighboring elevations equals the distance between neighboring spots on the original array, with the cross-sectional area of said elevations, however, being smaller than the spot area. In this way, the spot size in the stamping process is reduced. Staggered contact printing with different topological template dies makes it possible not only to copy the original arrays but also to reduce their size by several orders of magnitude. It is furthermore possible, by using dies with irregular predefined relief structures, to select particular desired molecule spots from an original array and combine them to a new copy array.

Advantageously, the use of topological dies makes it possible to obtain spot sizes of less than 10 μm, and in particular of less than 2 μm.

The demands on materials for the preparation of topological dies are the following: precise struturability, sealing contact to a suitable substrate (conformal contact), chemical and physical resistance to reaction conditions. Furthermore, in the case of small structures, the geometrical placing accuracy (alignment) which is characterized by accurate positionability and relative positional alignment must be ensured. By way of preference but not by way of limitation, the material used for the preparation of dies is an elastomer, for example polydimethylsiloxane (PDMS).

When using strong bases or halogenated hydrocarbons, such as, for example, chlorinated solvents, in the reaction, it is possible to use harder, albeit chemically more inert, materials for template preparation. In order to ensure uniform contact with the substrate surface, it is in this case possible to construct a hybrid structure made of a hard and chemically resistant core and soft contact areas made of elastic material. Examples of hard materials which may be used are glass, silicon, gold, silver, nickel or other metals and also various plastic materials. The contact areas of the topological structures may comprise an elastomer such as PDMS or other siloxanes, silicones, gum-like polymers, polyurethanes and other shapable elastic thermoplasts. In addition, chemical modifications may increase the chemical resistance of the die. Possible examples thereof are an increase in the degree of crosslinking of the polymer, glazing of the outer layer of the die, surface treatment by applying a thin protective layer made of a protective polymer or a metal, or other suitable chemical modifications.

Structured dies are prepared by defining master structures, for example in the form of a silicon wafer or as structured glass by means of classical photolithography and using them as casting mold for the liquid prepolymer. After curing, the elastic polymer can be removed in the desired three-dimensional form. For the preparation of structures from PDMS, the reader is referred to the following review article: Xia and Whitesides, Angew. Chem. Int. Ed. 37: 550-575 (1998).

PDMS is transparent for wavelengths down to the lower UV range. According to the invention, this may be utilized in order to ensure, for example, optical control of the positioning of the die relative to the substrate (alignment). Furthermore, particular light-sensitive reactions, for example of coupling molecules to the substrate surface, may be controlled by light.

According to the invention, the template die may be flat, roller-shaped, curved or convex or may have another shape suitable for contact printing methods.

Template molecules which may be used according to the invention are oligomers and polymers of the nucleotide class (DNA, RNA, PNA and their derivatives). Copy molecules which may be synthesized according to the invention by the methods described in a polymerization reaction on the template are DNA, RNA or aptamers and their, in particular nuclease-resistant, derivatives such as PNA or thioRNA and also proteins and peptides. The aptamers synthesized by this method which act as binding molecules analogous to antibodies may be used in various binding assays and, in some cases, functionally replace the antibody arrays whose production is expensive and complicated. Nucleotides modified by amino acid residues or other functional groups may also be used in RNA synthesis. It will also be possible to synthesize carbohydrate oligomers or other combinatorily synthesized compounds on a template on the die. According to the invention, any molecule arrays of the nucleotide class may be used as original arrays for copying a multiplicity of copy arrays.

The invention is not limited to polymerization reactions. The use of processing enzymes such as proteases, nucleases, kinases, transaminases, methylases, synthetases and other enzymes in the preparation of copy arrays according to the method described is also possible. Thus a molecule on the substrate may be processed by one or more separate stamping processes, it being possible, in particular, to introduce proteolysis, phosphorylation, alkylation, glycosylation, methylation or other chemical modifications.

The present invention may be used not just for preparing arrays with molecule groups arranged dot-like in the form of spots. Molecules which together form a dot matrix, a circular, helical, strip-shaped, linear or other geometric arrangement may also be copied on a template and be transferred to a substrate true to location and are thus part of the 5 invention.

The template dies described in the invention may be used not only as templates for the synthesis of molecules. In a further application, molecules may be isolated with the aid of template dies from ready-made complex mixtures and be arranged in a predefined pattern according to the location of template molecules. In this way it is possible, for example, to prepare antibody arrays from an antibody mixture such as blood serum very rapidly and easily by using a template die to which antigens have been attached. Conversely, proteins can be arranged on an array using an antibody template.

The invention furthermore comprises the use of molecule arrays prepared in this way for analytical purposes. Thus it is possible to use the arrays prepared according to the method described advantageously for a multiplicity of studies in medical and veterinary diagnostics, drug development, quality control of biological agents, forensics, the study of plant metabolites, analyses in the context of environmental protection, or in research and development.

In addition, the present invention relates to the apparatus and devices required for the described preparation of molecule arrays and also to kits which comprise the molecule array and the auxiliary substances required for the analysis thereof in said apparatus.

Further advantages, features and possible applications of the invention are described below on the basis of the exemplary embodiments with reference to the drawings in which:

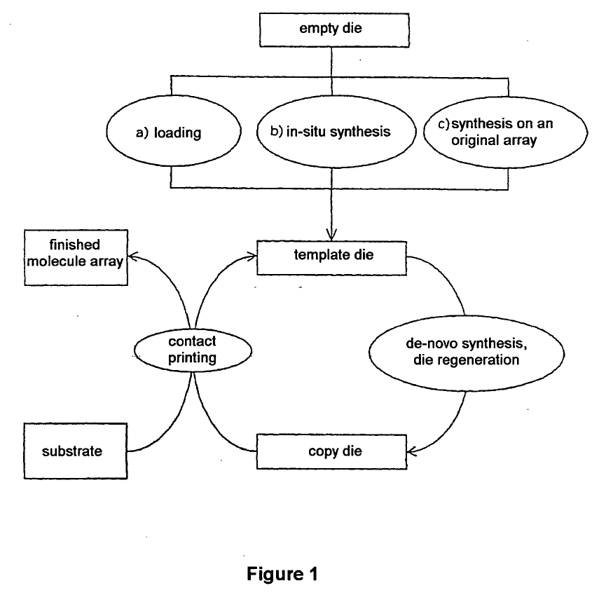

FIG. 1 depicts the basic principle of the method of the invention for preparing arrays,

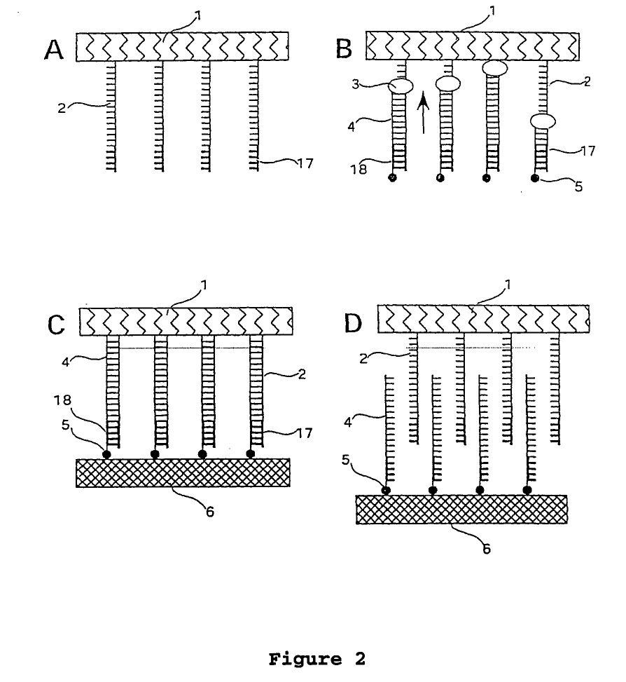

FIG. 2 depicts an exemplary embodiment of the method for preparing a DNA array,

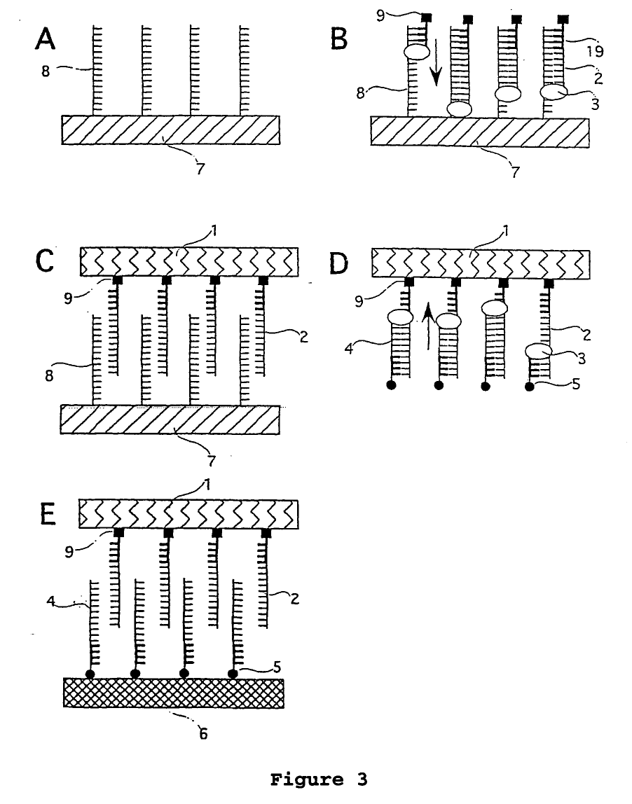

FIG. 3 depicts a variant of the preparation of a DNA array, starting from a differently prepared original DNA array,

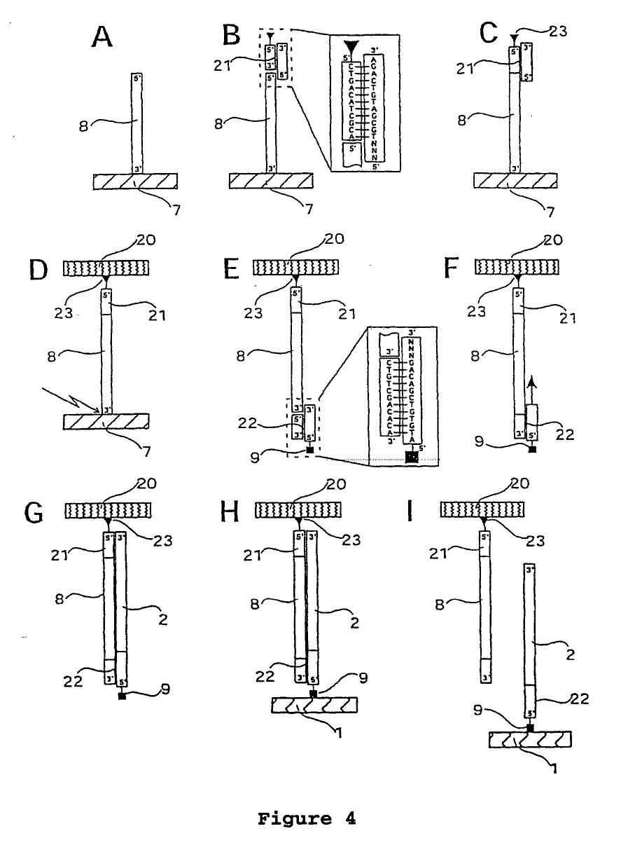

FIG. 4 depicts a variant of the preparation of a DNA array, starting from a differently prepared original DNA array in which the sequences are unknown and the molecules are attached to the array surface via their 3′ ends,

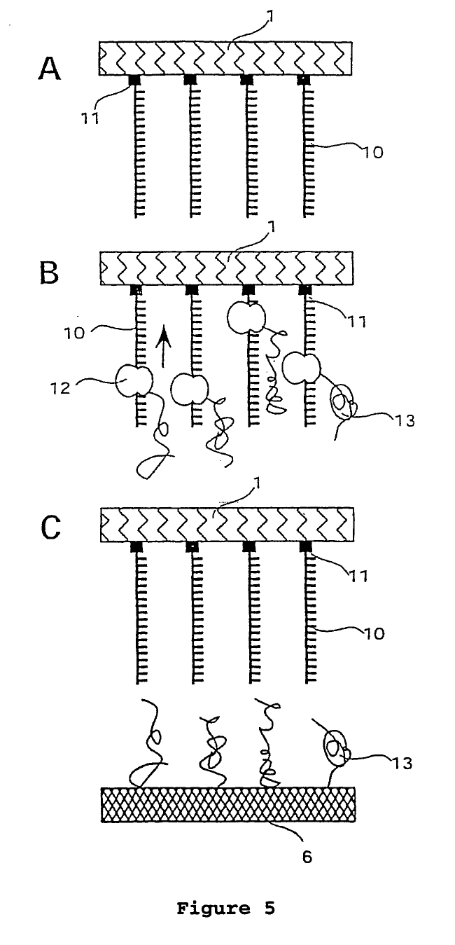

FIG. 5 depicts an exemplary embodiment of the method of the invention for preparing a protein array,

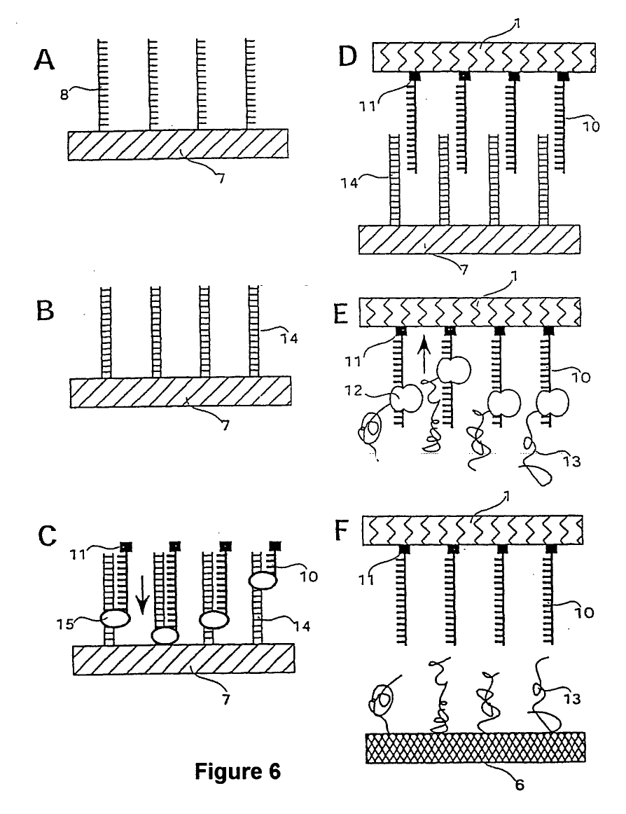

FIG. 6 depicts a variant of the preparation of a protein array, starting from a differently prepared original DNA array,

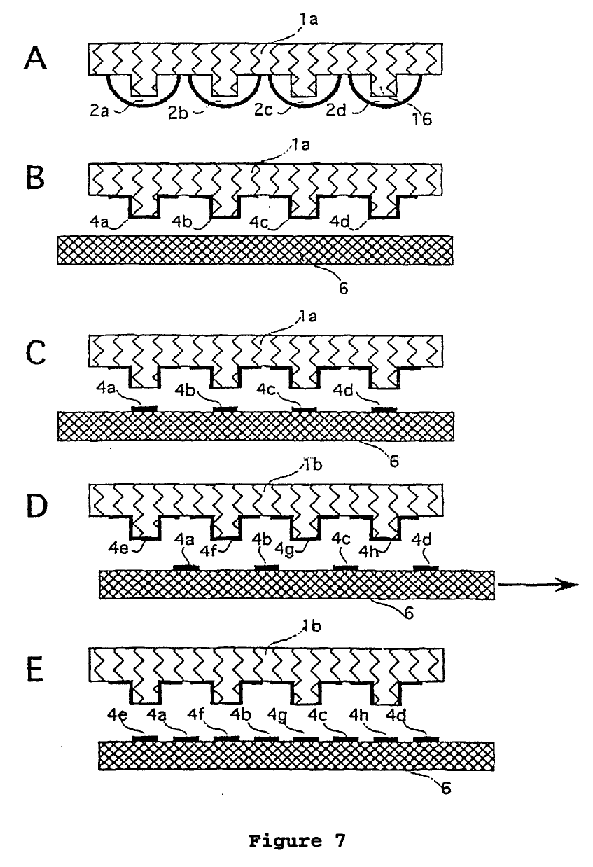

FIG. 7 depicts a variant of the method, in which it is possible, by using topological dies and by repeated laterally staggered contact printing, to obtain smaller spot sizes and a higher density than for application in drop form.

FIG. 1 depicts the basic principle of the method described in the invention. Firstly, a still empty die is coated with the template molecules. This may be carried out by any of the three following methods: a) loading the surface with ready-made macromolecules by an application in drop form, for example by means of printing pins or ink jet printers according to the prior art; b) by a sequential de-novo synthesis in situ of the macromolecules from individual components on the die surface according to the prior art; c) by a synthesis on the macromolecules of a differently synthesized original array which serve as templates for this synthesis according to the method of the invention. In this step the design of the template die determines the form and the contents of the array to be prepared.

In the second step, the copy molecules to be stamped are prepared by de-novo synthesis on the template molecules of the die. In the third step, the die is contacted with a substrate, thereby transferring the copy molecules to the substrate surface. The result is a finished molecule array with a defined spatial arrangement of spots, which is a mirror image of the template die and, respectively, a copy of the original array. After regeneration (de-novo synthesis of copy molecules), the template die may be used for a new preparation of arrays. The second and the third step may then be repeated several times so that it is possible to prepare a multiplicity of molecule arrays in a short time.

### EXAMPLES

a) Preparation of DNA Arrays

FIG. 2 depicts, in a simplified manner, the first preferred embodiment of the invention in which both template molecules and copy molecules are DNA sequences. In this example, the array molecules are prepared by in situ DNA polymerization on a substrate surface. A standard method may be used in order to apply the template DNA molecules 2 to the empty template die 1 (FIG. 2A). The DNA single strands whose length and sequence vary are complementary to the target sequences and are the templates for their synthesis. In a preferred variant, the template DNA is attached at its 5′ end to the die surface covalently or noncovalently, for example by means of an anchor, and comprises, by way of example and not by way of limitation, the following elements which may not necessarily be in the order indicated:

an anchoring group (e.g. an oligonucleotide of a defined sequence which is complementary to the sequence of an oligonucleotide on the die),

a spacer linking the DNA to the anchor,

the sequence complementary to the target DNA,

a primer binding sequence.

The primer binding sequence 17 which preferably comprises from eight to twenty nucleotides is identical in all template molecules on the die 1. Thus it is possible to replicate the DNA sequences on the entire array in a parallel reaction using a universal primer 18 which is complementary to the primer binding sequence. For this purpose, the die loaded with the template DNA is incubated in a polymerization cocktail known to the skilled worker, comprising, inter alia, DNA polymerase, nucleotides and primers. The copy DNA 4 is then synthesized by a DNA polymerase 3 in a primer extinction [sic] reaction on the template DNA 2 on the die 1 (FIG. 2B, arrow), said polymerization taking place on all spots at the same time. The freshly synthesized copy DNA remains, for the time being, hybridized to the DNA template at the site of the synthesis. In the subsequent contact printing process, all copy DNA molecules 4 are transferred to the target surface 6 simultaneously and in parallel (FIG. 2C). The DNA may bind to the substrate surface via an anchor 5, for example. Destabilization of the hydrogen bonds between the two DNA strands may be achieved, for example, by using detergents or salts, by changing the pH, or by increasing the temperature. When separating the die 1 from the substrate surface 6, the template molecules 2 remain on the die (FIG. 2D) and may be used as templates for further polymerization reactions in the next stamping cycles.

b) Copying Differently Prepared DNA Arrays

FIG. 3 depicts in a simplified manner a method for duplicating already existing DNA arrays. For this purpose, firstly a differently prepared original array 7 with the original DNA 8 placed thereupon (FIG. 3A) is used as template for preparing a regenerable DNA template die. If the DNA on the original array is not bound via its 3′ end to the array surface (this is the case, for example, for DNA arrays which are prepared by application in drop form) and its sequence is known, different primers 19 which are complementary to the 3′ ends of the original DNA sequences are constructed for the polymerization reaction. The template DNA 2 is synthesized on the original DNA 8 by a DNA polymerase 3 in a primary extension reaction on the surface of the original array 7 (FIG. 3B, arrow). The freshly synthesized array of template molecules 2 is then transferred to the still empty die 1 in a parallel step and bound, for example, with the aid of anchors 9 (FIG. 3C). The template DNA immobilized on the die may then be used, as described above, as template for the repeated synthesis of copy arrays (FIGS. 3D and 3E).

If original arrays in which unknown DNA sequences are attached via their 3′ ends to the surface need to be duplicated, then specially constructed adaptor elements may be used for polymerization instead of primers. FIG. 4 depicts a preferred embodiment variant for duplicating a DNA array in which the sequence is unknown and the DNA molecules 8 are attached via their 3′ ends to the array surface 7 (FIG. 4A). Arrays which are prepared by means of traditional solid-phase synthesis, for example in a phosphoramidite method or in a lithographic application method are constructed in this way, for example. In this case, first the orientation of the original DNA on the surface must be changed in order to render its 3′ end accessible to a polymerase. This may be achieved, for example, by using adaptor elements, cleavage at the DNA binding site and transfer to an auxiliary die. The first adaptor element 21 may, by way of example and not by way of limitation, have the following sequence:

5′-CTG-ACA-TCG-CA-3′
3′-A-GAC-TGT-AGC-GTN-N(N)-5′,

where N is any of the nucleotides A, T, G or C. These two to three permutated (“wobble”) nucleotides should make hybridization to the 5′ end of the DNA with unknown sequence possible (FIG. 4B). After ligation of the first adaptor 21 to the original DNA 8 (FIG. 4C), for example with the aid of a T4 ligase, the original DNA is transferred in a contact printing method to the empty auxiliary die 20 by a parallel and true-to-location absorption (FIG. 4D). It may be necessary here to separate the DNA from the array surface 7 either by cutting enzymatically, for example with restriction endonuclease, or by chemical cleavage, for example using a KOH solution in isopropanol (FIG. 4D, “lightning” arrow). Binding of the original DNA 8 to the surface of the auxiliary die 20 may be enabled by an anchor 23, for example a biotin which is attached to the first adaptor element. In this step, the DNA is attached via its 5′ end to the surface so that it is in a favorable orientation for polymerization. In the next step, a second adaptor element 22 which has been constructed according to the same principle but which does not necessarily have the same sequence, which sequence is, by way of example and not by way of limitation.

5′-CTG-TCG-ACA-CA-3′
3′-NNN-GAC-AGC-TGT-GTA-5′

is hybridized (FIG. 4E) and ligated (FIG. 4F) to the 3′ end of the original DNA 8. The second adaptor element comprises an anchor 9 for binding the DNA to the die surface and a primer binding sequence for hybridization of a universal primer for the subsequent synthesis of copy DNA. In the polymerization cocktail, comprising DNA polymerase and nucleotides, the template DNA 2 is then synthesized in a primer extension reaction on the original DNA 8, starting from the sequence of the second adaptor 22 (FIG. 4F, arrow, and 4G). The freshly synthesized template DNA 2 is transferred to the surface of the template die 1 by contact printing and bound via an anchor 9, for example (FIG. 4I). It is then possible to synthesize in a polymerization reaction as described in example a) the copy DNA on the template DNA on the template die and to transfer said copy DNA to the target surface by means of contact printing. In this and further stamping cycles, the polymerization reaction uses a universal primer which binds to any DNA molecules and which is complementary to the primer binding sequence of the first adaptor. In this case, the universal primer has the sequence

5′-CTG-ACA-TCG-CA-3′.

The application of the said method for copying DNA array with known sequences whose 3′ ends are coupled to the surface and arrays with unknown sequences whose 3′ ends are free are subexamples of the two cases set forth above. The implementation thereof follows from examples a) and b) and can be carried out by the skilled worker.

c) Preparation of Protein Arrays

The exemplary embodiment depicted in FIG. 5 is another preferred application of the described method for preparing protein arrays. In this case, molecule libraries of polypeptides are prepared on the substrate surface by in situ translation. The positional information is determined for each individual protein spot by the arrangement of the RNA coding therefor on the die surface.

The RNA molecules 10 are firstly applied to the die surface 1 by a standard method, for example by application in drop form, and bound covalently or noncovalently via an anchor 11, for example (FIG. 5A). The bound RNA molecules then serve as templates for the preparation of proteins. In order to increase the stability of said RNA molecules, chemically modified nuclease-resistant RNA molecules may be used. According to the prior art, these molecules may be, in particular, thio-RNA. For simplification purposes, any forms of translatable polynucleotides are referred to as RNA hereinbelow. Such molecules may be prepared synthetically, for example according to the prior art.

The sequence of the RNA molecules used as templates comprises, by way of example and not by way of limitation, the following elements which are not necessarily in the order indicated:

an anchoring group (e.g. an oligonucleotide of a defined sequence which is complementary to the sequence of an oligonucleotide on the die),

a sequence of the genetic elements required for initiation and continuation of an enzymic translation reaction, in particular a ribosomal binding site and a start codon, e.g. AUG,

a sequence encoding an oligopeptide (tag) which has high affinity for a binding molecule on the substrate surface,

the sequence of the protein to be printed,

a sequence which encodes an oligopeptide spacer which enables the full-length protein to leave the ribosome, without preliminary disintegration of the complex with the RNA, for example one or more repeats of a sequence encoding the oligopeptide (Gly)₄Ser,

in a preferred embodiment, the RNA does not end with a stop codon in order to prevent the dissolution of transcription complexes; alternatively, particular signals or chemical modifications on the RNA may be used.

The die loaded with the RNA is incubated in an in vitro-translation cocktail which comprises the components required for running a translation reaction. Such suspensions may be prepared, for example, from E. coli or rabbit reticulocytes, and are known to the skilled worker.

If long or complex polypeptide sequences need to be prepared, additional components, in particular chaperones, chaperonins or other substances supporting folding, such as, for example, glutathione, may be added to the in vitro-translation cocktail. This is particularly recommendable, if the polypeptide sequences are fragments of complex proteins which are not naturally folded in the cytoplasm of a cell. Typical examples thereof are members of the immunoglobulin superfamily, such as cell surface receptors, antibodies or T cell receptors.

The ribosome 12 stops synthesizing the polypeptide sequences 13 on the RNA, when the end of said RNA is reached. However, the translation complex does not disintegrate, since, in a preferred embodiment variant, the RNA does not contain a stop codon. The die 1 which now, in addition to the template RNA 10 also comprises the newly synthesized polypeptides 13 which are immobilized 5 at the site of their synthesis (FIG. 5B) is removed from the in vitro-translation cocktail and washed with physiological buffer.

The die 1 with the newly synthesized proteins 13 is then pressed onto the surface 6 to be stamped. According to the invention, an auxiliary substance may be used which destabilizes ribosomes or translation complexes, typically a chelator such as EDTA or EGTA. The complex of RNA 10 and protein 13 dissolves and proteins are transferred to the surface 6 to be stamped (FIG. 5C). After removing the die, the stamped surface has a coating of proteins which is a mirror image of the arrangement of the coding RNA on the substrate. Thus it is ensured that a signal generated at a particular site of this protein array by binding of an analyte can be unambiguously assigned to a protein with a particular sequence. The above-described method can be used to prepare advantageously arrays with peptides, proteins, antibodies or antibody fragments, in particular scFv fragments.

d) Translation of Differently Prepared DNA Arrays

FIG. 6 depicts an extension of the above-described example to the preparation of a protein array, starting from a differently prepared original array 7 whose surface carries the single-stranded DNA 8 (FIG. 6A). In this case, the double-stranded DNA 14 is first prepared in a polymerization reaction with the aid of a DNA polymerase according to the prior art (FIG. 6B). If the original DNA is not attached via its 3′ end to the array surface, the complementary strand may be synthesized, for example, in a primer extension reaction using a specially constructed primer. Said primer may comprise a sequence containing the signals required for transcription and translation. For example, the sequence of the T7 promoter may be used for transcription. The primer may be bound to the DNA strands either by using complementary sequences, if the original sequences are known, or by introducing permutated (“wobbled”) nucleotides, as described in example b). Alternatively, the sequence containing the signals required for transcription and translation may be attached directly to the 3′ end of the original DNA single strand by enzymic or chemical synthesis according to the prior art. If arrays with DNA molecules attached via their 3′ ends to the array surface are to be translated, the orientation of the latter first needs to be reversed. This may be carried out, for example, via an auxiliary die by means of specially constructed adaptor elements, as describe in example b).

In the next step, the RNA 10 to be applied is synthesized in a transcription reaction by an RNA polymerase on the double-stranded DNA template 14 (FIG. 6C). Alternatively, preparation of the double-stranded DNA and subsequent transcription may be dispensed with by hybridizing small RNA fragments with random sequences to the single-stranded original DNA and ligating them together to give an RNA strand complementary to the original DNA, using a T4 RNA ligase in a cocktail comprising individual nucleotides. Said T4 RNA ligase may also be used for attaching an anchor coupled to a nucleotide or an oligonucleotide to the 5′ end of the freshly synthesized RNA.

The freshly synthesized RNA 10 is then transferred to a die 1 and bound covalently or noncovalently, for example via an anchor 11, to the die surface (FIG. 6D). The die prepared in this way is further used for the repeatable synthesis and transfer of proteins to an array surface, as described above in example c) (FIGS. 6E and 6F). Using this method it is thus possible to copy over, transcribe and translate DNA arrays for the preparation of protein arrays. The described application might be used, for example, advantageously in EST microarrays (expressed sequence tags) for the analysis of sequences with unknown functions.

The described location-bound duplication according to the invention of also highly complex protein libraries allows a substantially less expensive preparation of protein arrays than with previous methods. The application of the method for preparing protein arrays is also suitable for improving the quality and simplifying the preparation of antibody arrays or arrays of functionally equivalent molecules such as anticalins, fibronectin variants, single-domain antibodies, affibodies or binding molecules based on other backbones. The antibodies or functionally equivalent molecules need not be prepared and purified individually prior to application to the substrate, but may be synthesized simultaneously and highly parallel in a joint reaction. In the method of the invention, only DNA which is substantially easier to purify and to control with respect to its quality must be prepared and purified individually.

The invention further comprises the use of protein arrays prepared in this way for analytical purposes. In an exemplary application, proteins of various infectious pathogens or else antibodies against pathogens may be applied to an array. Patients suffering from unspecified fever may then be examined, on the basis of a single blood or tissue sample, for a multiplicity of different pathogens simultaneously by using such a protein array.

e) Reducing the Spot Size and Increasing the Spot Density

FIG. 7 depicts in a simplified manner an application example of the described invention for preparing molecule arrays with a smaller spot size and a higher spot density than arrays prepared by application in drop form. Let us assume that an application in drop form can generate arrays with a spot diameter of 40 μm and a distance of 100 μm between the neighboring spots. In this example, using the method of the invention, a molecule array is prepared which has a spot diameter of 10 μm and a distance of 50 μm between the spots. For this purpose, in a first step, a topological die 1a is generated on which elevations 16 which have circular cross sections or are shaped in a different way and which have a diameter of 10 μm are located at a distance of 100 μm. Solutions of different template molecules 2a to 2d are applied by means of application in drop form to the elevations of the die in a 100 μm grid (FIG. 7A). After applying the template molecules and drying the spots, the corresponding copy molecules 4a to 4d are synthesized on this template array according to the above-described method (FIG. 7B). Next, the die is contacted with the substrate 6 (FIG. 7C). In this process, only the molecules located on the proximal area of said elevations are transferred to the substrate surface, since only these molecules come into direct contact with said substrate surface. This step thereby reduces the spot diameter from 40 μm to 10 μm. In the next stamping process, a new die 1b is used which, in this example, has the same topology but different molecule spots 4e to 4h (FIG. 7D). In order to achieve a higher spot density, the substrate 6 is moved here by 50 μm along its x-axis (FIG. 7D, arrow). During contact printing, the new spots 4e, 4f, 4g and 4h are placed on the substrate surface 6 exactly between the previously applied spots 4a, 4b, 4c and 4e at a distance of 50 μm (FIG. 7E). In the next two stamping processes, in each case 10 new dies are used and the substrate is moved relative to its starting position along its y- or x, y-axis by 50 μm. Altogether, four stamping processes with four different dies are carried out. In the example chosen, the spot density is quadrupled and the spot size is reduced to one quarter.

The present invention is not limited to the above-described exemplary embodiments. Rather, a plurality of modifications is possible which can be obtained by skilled workers and are therefore included within the scope of the present invention.

1. A method for preparing molecule libraries arranged in a defined pattern on a substrate, characterized by
preparation of at least one template die comprising a defined arrangement of template molecules,
true-to-location biochemical or chemical in situ synthesis of copy molecules on said template molecules located on the surface of said template die and serving as templates,
transfer of the synthesized copy molecules to a solid or polymeric substrate by means of contact printing, retaining the spatial arrangement of said copy molecules on the substrate,
reproducibility of the synthetic steps and transfer steps.

2. The method as claimed in claim 1, characterized in that the template molecules on the template die and the copy molecules on the substrate surface are arranged in the form of a dot matrix, a circular, helical, strip-shaped, linear or other geometric or stochastic structure.

3. The method as claimed in either of claims 1 and 2, characterized in that the molecule libraries synthesized on the substrate surface may be used for carrying out parallel binding reactions.

4. The method as claimed in any of claims 1 to 3, characterized in that the die comprises at least partially an elastic material, preferably polydimethylsiloxane.

5. The method as claimed in any of claims 1 to 4, characterized in that two or more different dies are used for preparing an array.

6. The method as claimed in any of claims 1 to 5, characterized in that the template molecules on the die are prepared by in situ synthesis on the molecules of a molecule array prepared in a different manner, which serve as templates.

7. The method as claimed in any of claims 1 to 5, characterized in that the template molecules on the die are prepared by chemical in situ synthesis or by in situ synthesis controlled by electric fields or light.

8. The method as claimed in any of claims 1 to 5, characterized in that the template molecules are applied to the die by application in drop form.

9. The method as claimed in any of claims 1 to 5, characterized in that the die is made of a material transparent for a particular wavelength and thus enables, via location-selective light conduction, photochemical reactions or near-field optical processes for binding or synthesizing the template molecules on the die surface to be carried out.

10. The method as claimed in any of claims 1 to 9, characterized in that the die is made of a material transparent for a particular wavelength and thus makes it possible to control relative positioning of said die on the substrate surface via location-selective light conduction.

11. The method as claimed in any of claims 1 to 10, characterized in that the template molecules are DNA, RNA or their nuclease-resistant derivatives such as PNA or thioRNA.

12. The method as claimed in any of claims 1 to 10, characterized in that the copy molecules are DNA, RNA or aptamers or their, in particular nuclease-resistant, derivatives such as PNA or thioRNA.

13. The method as claimed in any of claims 1 to 10, characterized in that the copy molecules are peptides, proteins, antibodies or antibody fragments, in particular scFv fragments, or molecules functionally equivalent thereto, in particular anticalins, fibronectin subdomains or individual antibody domains.

14. The method as claimed in any of claims 1 to 10, characterized in that the copy molecules are carbohydrates or combinatorially synthesized compounds.

15. The method as claimed in any of claims 1 to 14, characterized in that the copy molecules are modified after synthesis, in particular by means of proteolysis, phosphorylation, alkylation, glycosylation, methylation or chemical treatment.

16. The method as claimed in claim 15, characterized in that the copy molecules are modified in one or more stamping processes.

17. The method as claimed in any of claims 1 to 16, characterized in that the copy molecules comprise covalently or noncovalently bound anchors or chemical groups which mediate binding to the substrate surface to be printed.

18. The method as claimed in claim 17, characterized in that the substrate surface to be printed comprises the corresponding molecular counterpart to binding of the anchor or the substance reacting with the chemical group of said anchor.

19. The method as claimed in claim 18, characterized in that the anchor and its molecular counterpart on the substrate surface to be printed comprise, for example, in each case complementary nucleotide sequences or an antibody fragment and an antigen, or an S peptide and an S protein, or a biotin and a streptavidin, or a ligand and a receptor, or in each case vice versa.

20. The method as claimed in any of claims 1 to 19, characterized in that the surface of the die has topological structures in the form of a relief.

21. The method as claimed in claim 20, characterized in that the structured die comprises topological structures whose dimensions are smaller than those of the molecule spots applied to the die by means of a standard method, in particular application in drop form, and are preferably smaller than 10 μm, in particular smaller than 2 μm.

22. The method as claimed in claim 21, characterized in that molecule spots generated on the substrate are smaller than those generated on the structured die by means of a standard method, in particular application in drop form, and are preferably smaller than 10 μm, in particular smaller than 2 μm.

23. The method as claimed in either of claims 21 and 22, characterized in that repeated staggered contact printing produces an array having a higher spot density than the spot density generated on the structured die by means of a standard method, in particular application in drop form.

24. The method as claimed in claim 20, characterized in that the structured die comprises topological structures whose dimensions are smaller than the molecule spots on the molecule array prepared in a different manner and serving as template for molecule synthesis, and are preferably smaller than 10 μm, in particular smaller than 2 μm.

25. The method as claimed in claim 24, characterized in that molecule spots generated on the substrate are smaller than the spots on the molecule array prepared in a different manner and serving as template for molecule synthesis, and are preferably smaller than 10 μm, in particular smaller than 2 μm.

26. The method as claimed in either of claims 24 and 25, characterized in that repeated staggered contact printing generates an array having a higher density than on the molecule array prepared in a different manner and serving as template for molecule synthesis.

27. The method as claimed in any of claims 1 to 26, characterized in that it may be use for improving the quality and simplifying the preparation of protein and, in particular, of antibody arrays or of arrays of functionally equivalent molecules, in particular anticalins, fibronectin subdomains, antibody single chains or antibody fragments.

28. The method as claimed in claim 27, characterized in that the protein or antibody arrays or arrays of functionally equivalent molecules are used in the diagnostics of various pathogens.

29. An apparatus for preparing the dies and the molecule arrays according to a method as defined in claims 1 to 28, characterized in that individual steps are carried out semi-automatically or automatically.

30. A kit, comprising the essential substances for preparation of molecule arrays according to a method as defined in claims 1 to 28.

31. A kit, comprising the essential substances for carrying out binding assays on arrays prepared as claimed in any of claims 1 to 30.

32. A die, in particular template die, in particular for carrying out the method as claimed in any of claims 1 to 28.

33. The die as claimed in claim 32, characterized by at least one feature of the characterizing clauses of claims 1 to 28.
